SEQUENCE LISTING (12 sequences)

SEQ ID NO: 1 - RNA, 21 nt, Artificial Sequence
  Sequence is synthesized
ccuccgucguaguauucauuu

SEQ ID NO: 2 - RNA, 21 nt, Artificial Sequence
  Sequence is synthesized
uuggaggcagcaucauaagua

SEQ ID NO: 3 - RNA, 21 nt, Artificial Sequence
  Sequence is synthesized
gccuaagugauagugcaauuu

SEQ ID NO: 4 - RNA, 21 nt, Artificial Sequence
  Sequence is synthesized
uucggauucacuaucacguua

SEQ ID NO: 5 - RNA, 21 nt, Artificial Sequence
  Sequence is synthesized
gacgguuagaugagcuugauu

SEQ ID NO: 6 - RNA, 21 nt, Artificial Sequence
  Sequence is synthesized
uucugccaaucuacucgaacu

SEQ ID NO: 7 - RNA, 21 nt, Artificial Sequence
  Sequence is synthesized
gugagcgcuuuacauuagauu

SEQ ID NO: 8 - RNA, 21 nt, Artificial Sequence
  Sequence is synthesized
uucacucgcgaaauguaaucu

SEQ ID NO: 9 - RNA, 21 nt, Artificial Sequence
  Sequence is synthesized
gucagcguauaggugauguuu

SEQ ID NO: 10 - RNA, 21 nt, Artificial Sequence
  Sequence is synthesized
uucagucgcauauccacuaca

SEQ ID NO: 11 - RNA, 21 nt, Artificial Sequence
  Sequence is synthesized
gugugagucuccguugauauu

SEQ ID NO: 12 - RNA, 21 nt, Artificial Sequence
  Sequence is synthesized
uucacacucagaggcaacuau

(19) United States
(12) Patent Application Publication
(10) Pub. No.: US 2018/0155680 A1
(43) Pub. Date: Jun. 7, 2018

(54) ESTABLISHING PLURIPOTENCY IN MOUSE EMBRYONIC STEM CELLS

(71) Applicants: Hossein Baharvand, Tehran (IR); Seyedeh Nafiseh Hasani, Tehran (IR); Mehdi Totonchi, Tehran (IR); Hamid Gourabi, Tehran (IR)

(72) Inventors: Hossein Baharvand, Tehran (IR); Seyedeh Nafiseh Hasani, Tehran (IR); Mehdi Totonchi, Tehran (IR); Hamid Gourabi, Tehran (IR)

(73) Assignee: Royan Institute, Tehran (IR)

(21) Appl. No.: 15/821,645

(22) Filed: Nov. 22, 2017

Related U.S. Application Data
(63) Continuation of application No. 14/462,598, filed on Aug. 19, 2014, now abandoned.

(51) Int. Cl. C12N 5/0735

(52) U.S. Cl. CPC C12N 5/0606; C12N 2501/235; C12N 2501/15; C12N 2501/999

(57) ABSTRACT

A method for derivation and maintenance of pluripotency in mouse embryonic stem (mES) cells is disclosed. The method includes isolating mES cells from mouse embryos in a culture medium including an R2i compound, and culturing the mES cells in a medium including the R2i compound or a transforming growth factor beta (TGF-β) signaling pathway inhibitor. The R2i compound includes combination of a transforming growth factor beta (TGF-β) signaling pathway inhibitor and an extracellular signal-regulated kinases (ERK) signaling pathway inhibitor. This method can facilitate the homogeneous expression of pluripotency factors in embryonic stem cells.

### CROSS-REFERENCE TO RELATED APPLICATIONS

This application is a continuation of pending U.S. patent application Ser. No. 14/462,598, filed on Aug. 19, 2014, and entitled “Method for Derivation and Long-term Establishment of Ground State Pluripotent Embryonic Stem Cells,” the entire content of which is incorporated herein by reference.

### SPONSORSHIP STATEMENT

This application has been sponsored by Iran Patent Center, which does not have any rights in this application.

### TECHNICAL FIELD

The present disclosure generally relates to a method for derivation and maintenance of pluripotency in mouse embryonic stem cells using inhibitor molecules of a transforming growth factor beta (TGF-β) signaling pathway, and an extracellular signal-regulated kinases (ERK) signaling pathway inhibitor.

### BACKGROUND

Stem cells are undifferentiated cells that can be divided to produce more stem cells or differentiated into specialized cells. In developing organisms, embryonic stem cells can be differentiated into the various specialized cells such as derivatives of the ectoderm, endoderm and mesoderm germ layers. Embryonic stem cells can be cultured in laboratories with standard conditions. However, establishing pluripotency and identifying optimal cell culture conditions remain a significant challenge for long-term maintenance of embryonic stem cells in an undifferentiated state.

Conventional undefined culture conditions do not support embryonic stem cells derivation from most mouse strains. In these culture conditions, the stem cells are exposed to a conflict between the expression of pluripotency factors and differentiation lineage marker genes. Defined culture conditions for embryonic stem cells have been established regarding to identification of leukemia inhibitory factor (LIF) and bone morphogenetic protein 4 (BMP4). However, the derivation of embryonic stem cells from refractory strains and acquisition of optimal culture conditions for the undifferentiated state of embryonic stem cells remain unresolved.

In addition, laboratories have been able to use small molecules, such as small molecules for inhibiting the fibroblast growth factor 4 (FGF4) signaling pathway and glycogen synthase kinase 3 (GSK3) pathway, for the derivation and long-term maintenance of mouse embryonic stem cells. However, long-term stability of embryonic stem cells in such conditions is still being studied, as GSK3 inhibitors have also been shown to induce chromosomal instability.

Therefore, there is a need in the art for an efficient and reliable method for the derivation and long-term maintenance of embryonic stem cells which is not associated with instability in the embryonic stem cells. Furthermore, there is need in the art for a method which induces homogeneous expression of pluripotency factors in the embryonic stem cells, and facilitates the derivation of embryonic stem cells from different mouse strains with great efficiency.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a method for derivation and maintenance of pluripotency in mouse embryonic stem (mES) cells. The method may include isolating mES cells from mouse embryos in a culture medium including an R2i compound, and culturing the mES cells in a medium including the R2i compound or a transforming growth factor beta (TGF-β) signaling pathway inhibitor. The R2i compound may include a combination of a transforming growth factor beta (TGF-β) signaling pathway inhibitor and an extracellular signal-regulated kinases (ERK) signaling pathway inhibitor.

The above general aspect may include one or more of the following features. In one example, the TGF-β signaling pathway inhibitor includes a small molecule, or a siRNA molecule. In some cases, the small molecule includes one of SB431542, A83-01, or ALK5i. In another example, the siRNA molecule may be an inhibitory molecule against smad2 molecule, or smad3 molecule. According to some implementations, the SB431542 small molecule is present in the culture medium at a concentration ranging between about 2 μM and 10 μM. In another implementation, the A8301 small molecule is present in the culture medium at a concentration of about 0.5 μM. In one implementation, the ALK5i small molecule is present in the culture medium at a concentration of about 1 μM.

In some other implementations, the ERK signaling pathway inhibitor includes a small molecule. In one implementation, the ERK signaling pathway inhibitor is PD0325901. In some cases, the PD0325901 small molecule is present in the culture medium at a concentration of about 1 μM.

In another example, the mES cells are obtained from a mouse strain which is selected from a group consisting of NMRI strain, C57BL/6 strain, BALB/c strain, DBA/2 strain, and a hybrid of C57BL/6 strain and CD-1 strain. According to some implementations, the mouse embryos includes blastocysts, inner cell mass (ICM) cells, or single blastomere cells. In one implementation, the single blastomere cells are isolated from a 2-cell embryo, a 4-cell embryo, or an 8-cell embryo.

As another example, the culture medium may include a serum-free medium. For example, the serum-free medium can include one of N2B27 defined medium, or knock-out serum replacement (KoSR) supplemented medium. The culture medium can further include leukemia inhibitory factor (LIF). According to some implementations, the derivation and culturing the mES cells can occur in an adherent culture, or a suspension culture. The adherent culture includes a feeder-free culture in some cases. The mES cells are passaged over a period ranging between about 2 days and 3 days in the culturing step.

In some implementations, the derivation of mES cells from a mouse blastocyst includes removing zona pellucida from the mouse blastocyst, culturing zona-free blastocysts in a culture medium including the R2i compound with or without LIF, and dissociating cells of the cultured blastocyst to obtain mES cells. In one example, the R2i compound may be a combination of TGF-β signaling pathway inhibitor and ERK signaling pathway inhibitor.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a method for the derivation and maintenance of pluripotency in mouse embryonic stem (mES) cells in a culture medium including a TGF-β signaling pathway inhibitor and an ERK signaling pathway inhibitor, according to one or more aspects of the present disclosure.

FIG. 1B illustrates a method for the derivation and maintenance of pluripotency in mouse embryonic stem (mES) cells using an R2i compound and a TGF-β signaling pathway inhibitor, according to one or more aspects of the present disclosure.

FIG. 2 presents derivation efficiencies of mouse embryonic stem (mES) cell lines using a medium supplemented with an R2i compound with or without leukemia inhibitory factor (LIF), according to one or more aspects of the present disclosure.

FIG. 3A presents derivation efficiencies of mES cells from single blastomeres in different culture media, according to one or more aspects of the present disclosure.

FIG. 3B presents derivation efficiencies of mES cells from single blastomeres of NMRI and BALB/c mouse strains, according to one or more aspects of the present disclosure.

FIG. 4A is a graph showing mRNA fold changes of mouse embryonic stem (mES) cells in different cell culture conditions, according to one or more aspects of the present disclosure.

FIG. 4B is a graph showing mRNA fold changes of differentiated cells with respect to corresponding mouse embryonic stem (mES) cells, according to one or more aspects of the present disclosure.

FIG. 5 is a graph presenting a chromosomal integrity profile of mouse embryonic stem (mES) cells which were cultured in a medium supplemented with an R2i compound, according to one or more aspects of the present disclosure.

FIG. 6A is a graph showing mRNA fold changes of mouse embryonic stem (mES) cells which were cultured in different cell culture conditions, according to one or more aspects of the present disclosure.

FIG. 6B is a graph showing mRNA fold changes of mouse embryonic stem (mES) cells after knockdown of Smad2 using a siRNA, according to one or more aspects of the present disclosure.

FIG. 6C is a graph showing mRNA fold changes of mouse embryonic stem (mES) cells after knockdown of Smad3 using a siRNA in embryonic stem cells, according to one or more aspects of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Disclosed herein is a novel method for efficient and reliable derivation and long-term maintenance of mouse embryonic stem (mES) cells from blastocysts, inner cell mass (ICM) cells, or single blastomere cells from 2-, 4-, and 8-cell mouse embryos. The method uses small molecule inhibitors of the extracellular signal regulated kinase (ERK) signaling pathway and transforming growth factor β (TGF-β) signaling pathway. In some implementations, the method may include the use of leukemia inhibitory factor (LIF), though in other implementations the use of LIF may be omitted. By using a TGF-β signaling pathway inhibitor and an ERK signaling pathway inhibitor, the derivation and long-term maintenance of pluripotency in mES cells can be improved.

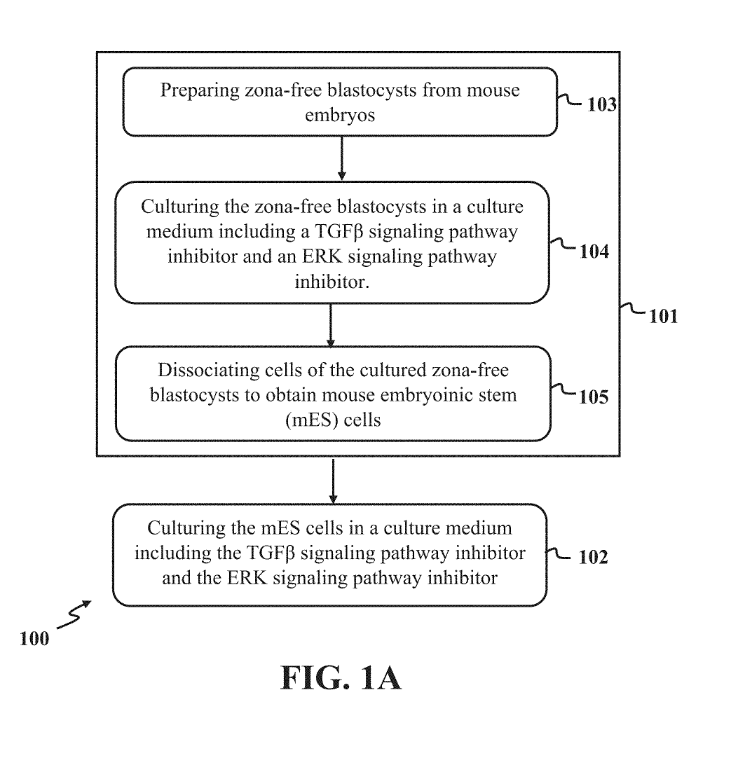

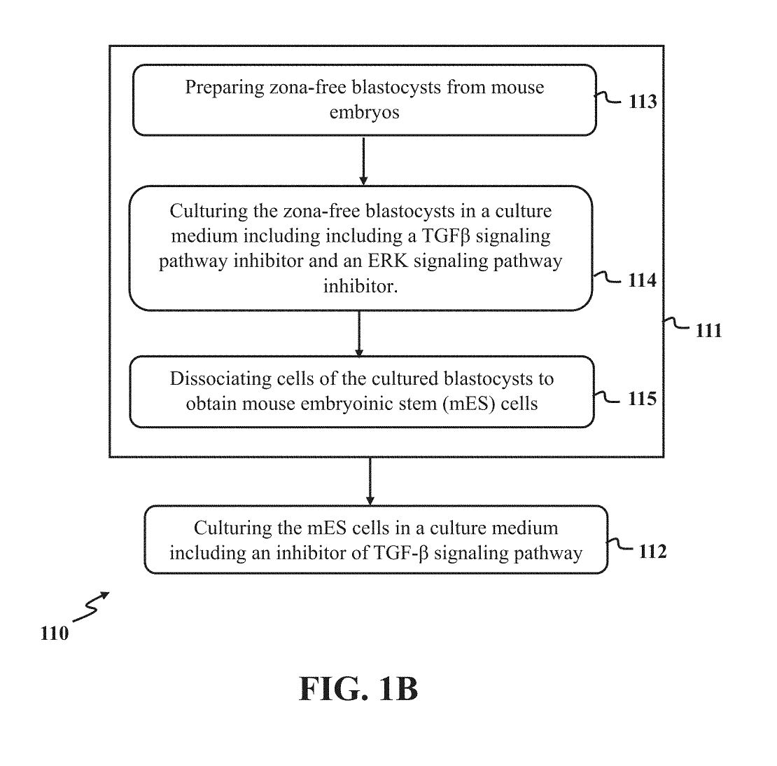

Referring first to FIG. 1A, one implementation of a method 100 for derivation and maintenance of pluripotency mES cells is depicted in a flow chart. In some implementations, the method may include the derivation of mES cells from mouse embryos using a culture medium including the TGF-β signaling pathway inhibitor and the ERK signaling pathway inhibitor (first step 101), and culturing the mES cells in a culture medium that includes the TGF-β signaling pathway inhibitor and the ERK signaling pathway inhibitor (second step 102). For purposes of this application, the term “R2i compound” refers to a combination of a TGF-β signaling pathway inhibitor and an ERK signaling pathway inhibitor according to the methods disclosed.

As noted above, the first step 101 can include the derivation of mES cells from the mouse embryos using a culture medium that includes the R2i compound. In some implementations, the derivation of the mES cells from the mouse embryos may further include preparing zona-free blastocysts from the mouse embryos (first substep 103), culturing the zona-free blastocysts in a culture medium including the R2i compound (second substep 104), and dissociating cells of the cultured zona-free blastocysts to obtain mES cells (third substep 105). Further details regarding these steps are provided below.

With respect to the first substep 103, in different implementations, the mouse embryos from which the mES cells are derived may include blastocysts, inner cell mass (ICM) cells, and/or single blastomere cells. The single blastomere cells may be isolated from 2-cell embryos, 4-cell embryos, or 8-cell embryos. In addition, in some implementations, the mouse embryos may be recovered by flushing the mouse embryos from the uteri or oviduct of mice. The mouse embryos may be from different mouse strains, for example, the NMRI strain, C57BL/6 strain, BALB/c strain, DBA/2 strain, or a hybrid of the C57BL/6 and CD-1 strains. After recovering the mouse embryos, zona-free blastocysts may be prepared by removing the zona-pellucida of the mouse embryo through treatment of the blastocysts with an acidic Tyrode's solution.

With respect to second substep 104, in some implementations, culturing zona-free blastocysts in the culture medium including the R2i compound can further involve the use of inner cell mass (ICM) cells for the derivation of mES cells. In such cases, the outer layer of the zona-free blastocysts which includes trophectodermal cells may be removed through an immunosurgery process. The isolated ICM cells may then be cultured in a medium supplemented with R2i compound for a period of time ranging between four and six days. In addition, in some implementations, culturing of the zona-free blastocysts or the ICM cells may be performed in an adherent culture or a suspension culture. Moreover, in one implementation, the adherent culture may include a feeder-free culture. Furthermore, the culture medium may include a serum-free medium, for example, N2B27 defined medium, or a knock-out serum replacement (KoSR) supplemented medium. The culture medium may further include leukemia inhibitory factor (LIF) in some cases.

In different implementations, the TGF-β signaling pathway inhibitor of the R2i compound may include a small molecule or a small inhibitory RNA (siRNA) molecule. The small molecule of the TGF-β signaling pathway inhibitor may include SB431542, A83-01, or ALK5i. In one implementation, the siRNA molecule of the TGF-β signaling pathway inhibitor may be an inhibitory molecule against Smad2 molecule, or Smad3 molecule. In addition, in some implementations, the SB431542 small molecule may be present in the culture medium at a concentration ranging between about 2 μM and about 10 μM. The A8301 small molecule may be present in the culture medium at a concentration of about 0.5 μM. The ALK5i small molecule may be present in the culture medium at a concentration of about 1 μM. Furthermore, according to some implementations, the ERK signaling pathway inhibitor of the R2i compound may include a small molecule. The small molecule of the ERK signaling pathway inhibitor may be PD0325901. The PD0325901 small molecule may be present in the culture medium at a concentration of about 1 μM.

Referring now to the third substep 105, following the culturing of the zona-free blastocysts for a period about four to six days, the cells may be dissociated by adding a trypsin solution to the cells for a period of time ranging between about 2 minutes and about 3 minutes. These cells can include blastocysts, single blastomeres, or ICM cells. The cells may then be cultured in new dishes. According to some implementations, the trypsin solution may be prepared by dissolving trypsin in ethylene diamine tetra acetic acid (EDTA) to form a trypsin solution with a concentration of about 0.05% (volume/volume). The trypsin solution may be neutralized by adding a mES cell medium including fetal bovine serum (FBS).

Following the substeps of first step 101, the method can further involve culturing the mES cells in a culture medium including the R2i compound in second step 102. According to one implementation, the culture medium may include a serum-free medium, for example, a N2B27 defined medium, or a knock-out serum replacement (KoSR) supplemented medium. The culture medium may further include leukemia inhibitory factor (LIF). According to one implementation, culturing of the mES cells may be performed in an adherent culture, or a suspension culture. Moreover, in some implementations, the adherent culture may include a feeder-free culture.

According to some implementations, the TGF-β signaling pathway inhibitor of the R2i compound may include a small molecule, or a siRNA molecule. The small molecule of the TGF-β signaling pathway inhibitor may include SB431542, A83-01, and/or ALK5i. In one implementation, the siRNA molecule of the TGF-β signaling pathway inhibitor may include an inhibitory molecule against the Smad2 molecule or Smad3 molecule.

In different implementations, the SB431542 small molecule may be present in the culture medium with a concentration ranging between about 2 μM and about 10 μM. In some implementations, the A8301 small molecule may be present in the culture medium at a concentration of about 0.5 μM. In one implementation, the ALK5i small molecule may be present in the culture medium with a concentration of about 1 μM. Similarly, according to some implementations, the ERK signaling pathway inhibitor of the R2i compound may include a small molecule. In one implementation, the small molecule of the ERK signaling pathway inhibitor may be PD0325901. The PD0325901 small molecule may be present in the culture medium with a concentration of about 1 μM.

Furthermore, during second step 102, after culturing the mES cells, in order to maintain their pluripotency, the mES cells may be passaged every 2 to 3 days. In order to passage the mES cells, the culture medium of wells including expanded mES cells may first be aspirated. The wells may also be washed with phosphate buffer saline (PBS) and a plurality of a trypsin solution can be added to the wells to dissociate the mES cells from the wells. In some implementations, following the addition of a trypsin solution to the mES, the mES cells may be incubated in an incubator. In some cases, the incubation can occur for between about 3 to 5 minutes, at a temperature of about 37° C. After the incubation, the trypsin solution may be neutralized by adding a medium containing serum to the mES cells.

As a result of adding the trypsin solution, in some implementations, the cells may be more readily dissociated into individual cells and small cell clumps when pipetted up and down between about 10 to 20 times. In some cases, the dissociated cells may then be centrifuged to remove the serum, and mixed with a fresh medium to form a suspension of mES cells. In one implementation, the suspension of mES cells may be cultured in a medium supplemented with the R2i compound. The culturing can occur with or without LIF as a suspension culture or an adherent culture.

Referring now to FIG. 1B, a method 110 for the derivation and maintenance of pluripotency in mouse embryonic stem (mES) cells is depicted in a flow chart. The method 110 may include a first step 111 of isolating mES cells from mouse embryos using a culture medium including R2i compound, and a second step 112 of culturing the mES cells in a culture medium including the TGF-β signaling pathway inhibitor.

In different implementations, the first step 111 includes isolating the mES cells from the mouse embryos using the culture medium including the R2i compound which is a combination of the TGF-β signaling pathway inhibitor and the ERK signaling pathway inhibitor. Thus, isolating the mES cells from the mouse embryos may further include in a first substep 113 of preparing zona-free blastocysts from the mouse embryos, a second substep 114 of culturing the zona-free blastocysts in the culture medium including the R2i compound, and a third substep 115 of dissociating cells of the cultured zona-free blastocysts to obtain mES cells.

As noted above, the first substep 113 of method 110 includes preparing zona-free blastocysts from mouse embryos. According to some implementations, the mouse embryos may include blastocysts, inner cell mass (ICM) cells, or single blastomere cells. The single blastomere cells may be isolated from 2-cell embryos, 4-cell embryos, or 8-cell embryos. The mouse embryos may be recovered by flushing the mouse embryos from uteri or oviduct of mice. In addition, in some implementations, the mouse embryos may be recovered by flushing the mouse embryos from uteri or oviduct of mice. The mouse embryos may be from different mouse strains, for example, the NMRI strain, C57BL/6 strain, BALB/c strain, DBA/2 strain, or a hybrid of the C57BL/6 strain and CD-1 strain. After recovering the mouse embryos, zona-free blastocysts may be prepared by removing zona-pellucida of mouse embryo through treating the blastocysts with an acidic Tyrode's solution.

The second substep 114 includes culturing zona-free blastocysts in the culture medium including the R2i compound. According to some implementations, inner cell mass (ICM) cells may be used for derivation of mES cells. Therefore, in some implementations, the outer layer of the zona-free blastocysts which includes trophectodermal cells may be removed through an immunosurgery process. The isolated ICM cells may be cultured in a medium supplemented with R2i compound for a period of time between about 4 days and 6 days. In one implementation, the culture medium may include a serum-free medium, for example N2B27 defined medium, or knock-out serum replacement (KoSR) supplemented medium. The culture medium may further include leukemia inhibitory factor (LIF). Furthermore, in some implementations, culturing of the zona-free blastocysts may be performed in an adherent culture, or a suspension culture. The adherent culture may include a feeder-free culture. The zona-free blastocysts may be cultured for a period between 4 days and 6 days.

According to some implementations, the TGF-β signaling pathway inhibitor may include a small molecule, or a siRNA molecule. The small molecule of the TGF-β signaling pathway inhibitor may include SB431542, A83-01, and/or ALK5i. In an exemplary embodiment, the siRNA molecule of the TGF-β signaling pathway inhibitor may be an inhibitory molecule against Smad2 molecule, or Smad3 molecule. In addition, in some implementations, the SB431542 small molecule may be present in the culture medium at a concentration ranging between about 2 μM and about 10 μM. The A8301 small molecule may be present in the culture medium at a concentration of about 0.5 μM in one implementation. In some implementations, the ALK5i small molecule may be present in the culture medium at a concentration of about 1 μM. According to some implementations, the ERK signaling pathway inhibitor of the R2i compound may include a small molecule. The small molecule of the ERK signaling pathway inhibitor may include PD0325901 in one implementation. The PD0325901 small molecule may be present in the culture medium at a concentration of about 1 μM.

The third substep 115 includes dissociating the ICM cells of the cultured zona-free blastocysts to obtain mES cells. In some implementations, after culturing the zona-free blastocysts for a period ranging between about 4 to 6 days, the cells may be dissociated. These cells can include the blastocysts, the single blastomeres, or the ICM cells. In one implementation, the cells are dissociated by adding a trypsin solution to the cells for a period ranging between about 2 minutes and about 3 minutes, and then the cells may be cultured in new dishes. In some implementations, the trypsin solution may be prepared by dissolving trypsin in ethylene diamine tetra acetic acid (EDTA) to form a trypsin solution with a concentration of about 0.05% (volume/volume). In one implementation, the trypsin solution may be neutralized by adding a mES medium including fetal bovine serum (FBS).

Following each of the three substeps that comprise the first step 111, the method 110 further involves the second step 112 culturing the mES cells in the culture medium including the TGF-β signaling pathway inhibitor. According to an implementation, the culture medium may include a serum-free medium, for example N2B27 defined medium, or knock-out serum replacement (KoSR) supplemented medium. The culture medium may further include leukemia inhibitory factor (LIF). In one implementation, culturing of the mES cells may occur in an adherent culture, or a suspension culture. The adherent culture may include a feeder-free culture.

Furthermore, in some implementations, the TGF-β signaling pathway inhibitor may include a small molecule, or a siRNA molecule. The small molecule of the TGF-β signaling pathway inhibitor may include SB431542, A83-01, or ALK5i. In one implementation, the siRNA molecule of the TGF-β signaling pathway inhibitor may be an inhibitory molecule against Smad2 molecule, or Smad3 molecule. In addition, in some implementations, the SB431542 small molecule may be present in the culture medium at a concentration ranging between about 2 μM and about 10 μM. The A8301 small molecule may be present in the culture medium at a concentration of about 0.5 μM in one implementation. In some implementations, the ALK5i small molecule may be present in the culture medium at a concentration of about 1 μM.

Referring back to second step 112, after culturing the mES cells, the mES cells may be passaged every 2 or 3 days to help maintain their pluripotency. In order to passage the mES cells, the culture medium of wells including the expanded mES cells and the TGF-β signaling pathway inhibitor may be aspirated. In some implementations, the wells may be washed with phosphate buffer saline (PBS) and a plurality of a trypsin solution added to the wells to dissociate the mES cells from the wells. In some implementations, after a trypsin solution has been added to the mES, the mES cells may be incubated in an incubator for a period ranging between about 3 minutes to 5 minutes at a temperature of about 37° C. After incubation, in one implementation, the trypsin solution may be neutralized by adding a medium containing serum to the mES cells.

In different implementations, as a result of adding the trypsin solution, the cells may be more readily dissociated into individual cells and small cell clumps by pipetting up and down between about 10 times to 20 times. The dissociated cells may be centrifuged to remove the serum, and then mixed with a fresh medium to form a suspension of mES cells. In some implementations, the suspension of mES cells may be cultured in a medium supplemented with the TGF-β signaling pathway inhibitor and with and without LIF as a suspension culture or an adherent culture.

### EXAMPLES

The following examples describe some implementations of the method for the derivation and maintenance of pluripotency in mES cells using the R2i compound of the present disclosure. The following examples further describe characterization of the mES cells which were obtained by the present method as well as a conventional method for derivation and maintenance pluripotency in mES cells for purposes of comparison.

In the conventional method for derivation and maintenance of pluripotency in mES cells, different small molecules for inhibiting fibroblast growth factor 4 (FGF4) signaling pathway and glycogen synthase kinase 3 (GSK3) pathway have been used. The small molecules of the conventional method have been referred to as “2i compound”. The 2i compound includes combinations of 1 μM of PD0325901 for inhibiting ERK signaling pathway and 3 μM of CHIR99021 for inhibiting GSK3 signaling pathway.

The 2i compound can cause suppression of endogenous differentiation-inducing signaling. Therefore, mES cell derivation can occur throughout different rodent strains using a cell culture medium supplemented with the 2i compound. In addition, the mES cells showed more homogeny in 2i than serum. Thus, the subpopulations and cell-to-cell nonconformity of Nanog and Rex-1 expression are reduced when using the 2i compound. However, the 2i compound causes GSK3 pathway inhibition and leads to different chromosomal abnormalities. As noted earlier the present application is directed to overcoming the shortcomings of the conventional methods. In the following Examples, the R2i compound, here including 1 μM of PD0325901 and 10 μM of SB431542, is used in the derivation and long-term maintenance of the mES cells.

### Example 1: Establishing Pluripotency in Mouse Embryonic Stem Cells from Blastocysts

In EXAMPLE 1, pluripotency was established in mouse embryonic stem (mES) cells. Mice from different strains were maintained on a 12-hour light/dark regimen. The mice were superovulated with a standard protocol, and embryos with 2, 4, and 8 cells were captured 44, 54, and 68 hours after injecting human chorionic gonadotropin (hCG) hormone to Fla, DBA/2, and BALB/c mouse strains. The mouse embryos were recovered by flushing uteri for collecting the blastocysts or by flushing oviduct for early cleavage embryos.

Derivation of mES cells at a blastocyst stage was accomplished by plating zona-free 3.5-day blastocysts on gelatin-coated plates including N2B27 medium with and without leukemia inhibitory factor (LIF) and supplemented with the R2i compound. The R2i compound was a combination of 1 μM of PD0325901 and 10 μM of SB431542.

Five to seven days after blastocysts plating, the cells were dissociated using a trypsin solution with a concentration of 0.05% trypsin (volume/volume). The trypsin solution was then neutralized by a mES cell medium containing FBS.

The dissociated mES cell solution was centrifuged to remove the FBS, and a plurality of dissociated mES cells were plated on gelatin-coated plates including N2B27 medium with and without LIF and supplemented with the R2i compound for cultivation of the mES cells as an adherent layer. The R2i compound was a combination of 1 μM of PD0325901 and 10 μM of SB431542. The fresh N2B27 medium was replaced to allow removal of the remnant FBS after adhesion of the mES cells, which occurred usually between 2 hours and 3 hours after plating the mES cells.

Following this step, the derived mES cells—irrespective of the way of derivation—were cultured on gelatin-coated plates including N2B27 medium supplemented with the R2i compound without MEF feeder layer. The R2i compound was a combination of 1 μM of PD0325901 and 10 μM of SB431542. In order to maintain the mES pluripotency, the mES cells were passaged every 2 days. Generally, one third of the cultured mES cells were passaged in every culture batch.

The culture medium of wells including expanded mES cells was first aspirated, and the wells were then washed with phosphate buffer saline (PBS). Following this step, 100 μM of trypsin/EDTA solution with a concentration of 0.05% w/v (weight/volume) was added to the wells, and the mES cells were incubated in an incubator at a temperature of about 37° C. for 3 minutes.

A medium containing fetal bovine serum (FBS) was subsequently added to the wells containing mES cells to inactive and neutralize the activity of trypsin. As a result of the trypsin activity, the mES cells dissociated readily into individual cells and small cell clumps by pipetting up and down with a 1000 pi pipette tip between about 10 times to 20 times. This lead to the formation of a mES cell suspension. The mES cell suspension was then centrifuged to remove the FBS.

After centrifugation, pellet of mES cell was mixed with N2B27 medium, and then transferred to bacterial dishes containing N2B27 medium supplemented with the R2i compound. This occurred with and without LIF for cultivation as suspension. Alternatively, the mES cells were passed on gelatin-coated plates containing N2B27 medium supplemented with the R2i compound, with and without LIF for cultivation as adherent layer.

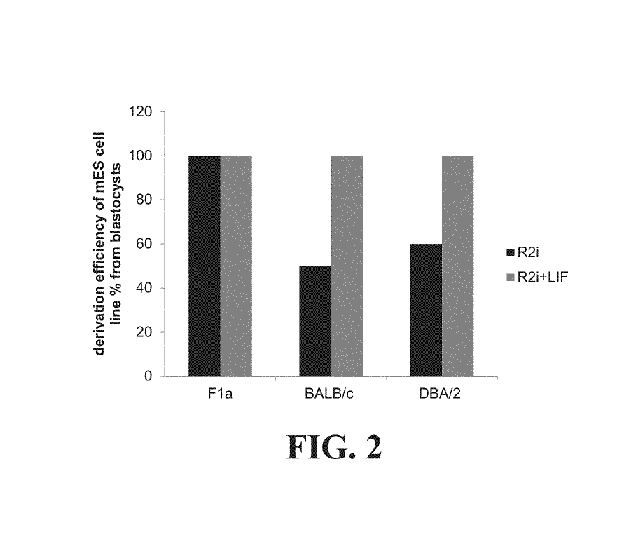

Referring to FIG. 2, derivation efficiencies of mouse embryonic stem (mES) cell lines using a medium supplemented with the R2i compound with and without leukemia inhibitory factor (LIF) are presented, according to one or more aspects of the present disclosure. In FIG. 2, the derivation efficiencies of mES cell lines of Fla, DBA/2, and BALB/c mouse strains using N2B27 medium supplemented with the R2i compound and LIF are 100% and higher than the derivation efficiencies of mES cell lines using N2B27 medium supplemented with the R2i compound without LIF.

### Example 2: Establishing Pluripotency in Mouse Embryonic Stem Cells Using Single Blastomeres

In EXAMPLE 2, the effect of the R2i compound on the derivation and maintenance of pluripotency in mES cell from single blastomeres was investigated. The R2i compound increases the development of embryonic cleavage and clonally propagation of mES cells from single blastomeres. In order to investigate the possibility that the R2i compound attains naive pluripotency from early cleavage stage mouse embryos, the mouse embryonic stem (mES) cells derivation from single blastomeres at different developmental stages was analyzed.

In an initial step, in order to exclude the probability that N2B27 basal medium induces a destructive effect on the embryonic cleavage, the embryo development of NMRI mouse strain from 2-cell stage towards intact hatched blastocyst in the droplets of different media was analyzed.

The media used included KSOM (potassium-supplemented simplex optimised medium) as a conventional cleavage medium, serum+ LIF, and N2B27 medium alone or supplemented with the 2i compound or the R2i compound. It was found that N2B27 medium is equal to KSOM in progressing early embryo development. The addition of the R2i compound to the N2B27 medium caused most of the early mouse embryos to gain the potential to thrive from 2-cell stage to intact hatched blastocysts significantly more than other media.

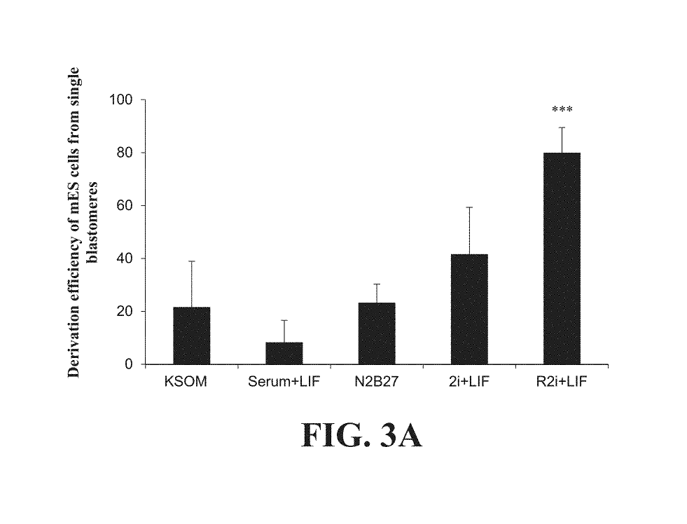

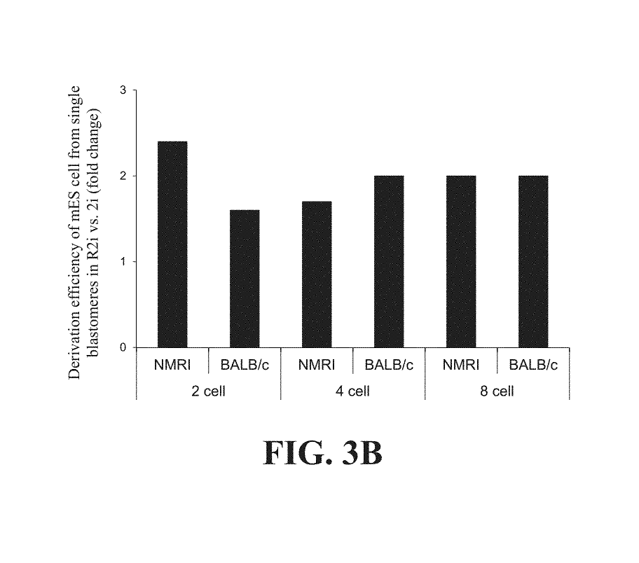

FIG. 3A presents derivation efficiencies of mES cells from single blastomeres in different culture media, according to one or more aspects of the present disclosure. As shown in FIG. 3A, the N2B27 medium is similar to KSOM medium, and both media allow progressive development of early embryos. In order to investigate the effect of the R2i compound on derivation and maintenance of pluripotency in mES cells from single blastomeres, mice from different strains were maintained on a 12-hour light/dark regimen. The mice were superovulated with a standard protocol, and early-cleavage embryos with 2, 4, and 8 cells were captured 44, 54, and 68 hours after injecting human chorionic gonadotropin (hCG) hormone to out-bred NMRI or inbred BALB/c mice, and the early-cleavage mouse embryos were recovered by flushing oviduct.

The zona-free blastocysts were then plated on gelatin-coated plates, and single blastomeres of the zona-free blastocysts with 2, 4, and 8 cells were separated by gentle pipetting of the zona-free blastocysts on fresh N2B27 medium. The single blastomeres from 2-, 4-, and 8-cell stage embryos of NMRI and BALB/c strains were subsequently evaluated for mES cell derivation.

The single blastomeres of each strain were transferred to mouse embryonic fibroblast (MEF)-coated 96-well plates containing N2B27 medium with LIF in the presence of FBS serum, the 2i compound, or the R2i compound. The R2i compound was a combination of 1 μM of PD0325901 and 10 μM of SB431542, or with the 2i compound.

In order to maintain the mES pluripotency, the mES cells were passaged every 2 days as follows. Generally, about one third of the cultured mES cells were passaged in every culture batch. At first, the culture medium of wells including expanded mES cells was aspirated, and the wells were washed with phosphate buffer saline (PBS). Following this step, 100 μM of trypsin/EDTA solution with a concentration of 0.05% w/v (weight/volume) was added to the wells, and the mES cells were incubated in an incubator at a temperature of about 37° C. for 3 minutes.

Subsequently, a medium containing fetal bovine serum (FBS) was added to the wells containing mES cells to deactivate and neutralize the activity of trypsin. As a result of trypsin activity, the mES cells were dissociated readily into individual cells and small cell clumps by pipetting up and down between about 10 times to 20 times with a 1000 pi pipette tip to form a mES cell suspension. The mES cell suspension was centrifuged to remove the FBS.

After centrifugation, pellet of mES cell was mixed with N2B27 medium. This mixture was then transferred to bacterial dishes containing N2B27 medium supplemented with the R2i compound, or with the 2i compound, both with and without LIF for cultivation as suspension. Alternatively, the mES cells were passed on gelatin-coated plates containing N2B27 medium supplemented with the R2i compound or with the 2i compound, with and without LIF for cultivation as an adherent layer. Some data regarding the derivation of mES cells of NMRI mouse strain from single blastomeres of 2-, 4-, and 8-cell embryos on gelatin-coated plates containing N2B27 medium in presence of FBS is presented in TABLE 1 below.

TABLE 1
Derivation of mES cells from single
blastomeres of NMRI mouse strain

| Cleavage stage | | Developed mES cell lines (%) | |
|---|---|---|---|
| Replicate | Number of embryos | 2i | R2i |
| 2-cell | | | |
| 1 | 15 | 0 | 0 |
| 2 | 24 | 0 | 0 |
| 4-cell | | | |
| 1 | 12 | 0 | 0 |
| 2 | 8 | 0 | 0 |
| 8-cell | | | |
| 1 | 26 | 0 | 0 |
| 2 | 16 | 0 | 0 |

The division ability of single blastomeres is usually reduced or lost on gelatin-coated plates. Therefore, referring to TABLE 1 above, in such a condition none of the single blastomere cells could attain pluripotency in N2B27 medium in the presence of FBS.

For purposes of comparison, data regarding the derivation efficiencies of mES cells of NMRI mouse strain from single blastomeres of 2-cell embryos of NMRI mouse strain on MEF feeder layer in N2B27 medium containing the 2i compound and the R2i compound is presented in Table 2 below.

TABLE 2
Derivation efficiency of mES cells from single blastomeres
of 2-cell embryos of NMRI mouse strain
NMRI Strain

| 2i | | | R2i | | |
|---|---|---|---|---|---|
| | Number of | Number of | | Number of | Number of |
| Number of | dissociated | established mES | Number of | dissociated | established mES |
| embryos | blastomeres | cell lines | embryos | blastomeres | cell lines |
| 1 | 2 | 0 | 1 | 2 | 0 |
| 2 | 2 | 2 | 2 | 2 | 2 |
| 3 | 2 | 0 | 3 | 2 | 2 |
| 4 | 2 | 0 | 4 | 2 | 2 |
| 5 | 2 | 0 | 5 | 2 | 0 |
| 6 | 2 | 2 | 6 | 2 | 2 |
| 7 | 2 | 0 | 7 | 2 | 1 |
| 8 | 2 | 1 | 8 | 2 | 2 |
| 9 | 2 | 1 | 9 | 2 | 2 |
| 10 | 2 | 1 | 10 | 2 | 0 |
| 11 | 2 | 0 | 11 | 2 | 2 |
| 12 | 2 | 0 | 12 | 2 | 0 |
| 13 | 2 | 0 | 13 | 2 | 2 |
| 14 | 2 | 0 | 14 | 2 | 1 |
| 15 | 2 | 1 | 15 | 2 | 0 |
| 16 | 2 | 0 | 16 | 2 | 0 |
| 17 | 2 | 0 | 17 | 2 | 2 |
| Total | 34 | 8 | Total | 34 | 20 |

Total efficiency of established mES cell lines (%)

| From total | From dissociated | From total | From dissociated |
|---|---|---|---|
| embryos | blastomeres | embryos | blastomeres |
| 35 | 24 | 65 | 59 |

In Table 3 below, derivation efficiencies of mES cell lines from single blastomeres of 4-cell embryos of NMRI mouse strain on MEF feeder layer in N2B27 medium containing the 2i compound and the R2i compound are presented.

TABLE 3
Derivation efficiency of mES cell lines from single
blastomeres of 4-cell embryos of NMRI strain
NMRI Strain

| 2i | | | R2i | | |
|---|---|---|---|---|---|
| | Number of | Number of | | Number of | Number of |
| Number of | dissociated | established mES | Number of | dissociated | established mES |
| embryos | blastomeres | cell lines | embryos | blastomeres | cell lines |
| 1 | 3 | 1 | 1 | 3 | 2 |
| 2 | 3 | 2 | 2 | 3 | 1 |
| 3 | 3 | 0 | 3 | 3 | 2 |
| 4 | 3 | 0 | 4 | 3 | 2 |
| 5 | 3 | 1 | 5 | 3 | 0 |
| Total | 15 | 4 | Total | 15 | 7 |

Total efficiency of established mES cell lines (%)

| From total | From dissociated | From total | From dissociated |
|---|---|---|---|
| embryos | blastomeres | embryos | blastomeres |
| 60 | 27 | 80 | 47 |

Referring next to Table 4 below, derivation efficiencies of mES cell lines from single blastomeres of 4-cell embryos of BALB/c mouse strain on MEF feeder layer in N2B27 medium containing the 2i compound and the R2i compound are presented.

TABLE 4
Derivation efficiency of mES cell lines from single
blastomeres of 4-cell embryos in BALB/c strain
BALB/c Strain

| 2i | | | R2i | | |
|---|---|---|---|---|---|
| | Number of | Number of | | Number of | Number of |
| Number of | dissociated | established mES | Number of | dissociated | established mES |
| embryos | blastomeres | cell lines | embryo | blastomeres | cell lines |
| 1 | 3 | 2 | 1 | 4 | 2 |
| 2 | 3 | 0 | 2 | 3 | 1 |
| 3 | 3 | 1 | 3 | 3 | 3 |
| 4 | 4 | 2 | 4 | 3 | 1 |
| 5 | 3 | 2 | 5 | 4 | 2 |
| 6 | 3 | 0 | 6 | 4 | 3 |
| 7 | 4 | 0 | 7 | 3 | 3 |
| Total | 23 | 7 | Total | 24 | 15 |

Total efficiency of established mES cell lines (%)

| From total | From dissociated | From total | From dissociated |
|---|---|---|---|
| embryos | blastomeres | embryos | blastomeres |
| 57 | 30.5 | 100 | 62.5 |

Next, as shown in Table 5 below, derivation efficiencies of embryonic stem cell lines from single blastomeres of 8-cell embryos of NMRI mouse strain on MEF feeder layer in N2B27 medium containing the 2i compound and the R2i compound are presented.

TABLE 5
Derivation efficiency of mES cell lines from single
blastomeres of 8-cell embryos in NMRI strain
NMRI Strain

| 2i | | | R2i | | |
|---|---|---|---|---|---|
| | Number of | Number of | | Number of | Number of |
| Number of | dissociated | established mES | Number of | dissociated | established mES |
| embryos | blastomeres | cell lines | embryo | blastomeres | cell lines |
| 1 | 8 | 0 | 1 | 8 | 3 |
| 2 | 8 | 2 | 2 | 8 | 5 |
| 3 | 8 | 4 | 3 | 8 | 4 |
| Total | 24 | 6 | Total | 24 | 12 |

Total efficiency of established mES cell lines (%)

| From total | From dissociated | From total | From dissociated |
|---|---|---|---|
| embryos | blastomeres | embryos | blastomeres |
| 67 | 25 | 100 | 50 |

In Table 6 below, derivation efficiencies of embryonic stem cell lines from single blastomeres of 8-cell embryos of BALB/c mouse strain on MEF feeder layer in N2B27 medium containing the 2i compound and the R2i compound are presented.

TABLE 6
Derivation efficiency of mES cell lines from single
blastomeres of 8-cell embryos in BALB/c strain
BALB/c Strain

| 2i | | | R2i | | |
|---|---|---|---|---|---|
| | Number of | Number of | | Number of | Number of |
| Number of | dissociated | established mES | Number of | dissociated | established mES |
| embryo | blastomeres | cell lines | embryo | blastomeres | cell lines |
| 1 | 8 | 2 | 1 | 8 | 3 |
| 2 | 8 | 0 | 2 | 8 | 2 |
| 3 | 8 | 4 | 3 | 8 | 3 |
| 4 | 8 | 0 | 4 | 8 | 5 |
| 5 | 8 | 4 | 5 | 8 | 4 |
| 6 | 8 | 1 | 6 | 8 | 5 |
| Total | 48 | 11 | Total | 48 | 22 |

Total efficiency of established mES cell lines (%)

| From total | From dissociated | From total | From dissociated |
|---|---|---|---|
| embryos | blastomeres | embryos | blastomeres |
| 67 | 23 | 100 | 46 |

With reference to TABLES 1-6 above, it can be seen that the efficiency of mES cell derivation in mediums supplemented with the R2i compound showed a nearly 2-fold increase relative to mediums supplemented with the 2i compound.

For purposes of clarity, FIG. 3A illustrates derivation efficiencies of mES cells from single blastomeres of NMRI and BALB/c mouse strains in a bar graph, according to one or more aspects of the present disclosure. In addition, in FIG. 3B, the derivation efficiency of mES cell from single blastomeres in medium supplemented with the R2i compound and LIF is shown as being nearly two-fold greater than the derivation efficiency of mES cell in medium supplemented with the 2i compound and LIF.

### Example 3: Establishing Pluripotency in Mouse Embryonic Stem Cells Using ICM Cells

In EXAMPLE 3, the effect of the R2i compound on the derivation and maintenance of pluripotency in mES cell using inner cell mass (ICM) cells was investigated. Several zona-free 3.5-day blastocysts of C57BL/6 (Oct4APE: GFP+ or OG2)×CD-I mouse strain were initially cultured on gelatin-coated 96-well plates containing N2B27 medium supplemented with the R2i compound (as test groups) and on gelatin-coated 96-well plates containing N2B27 medium supplemented with FBS serum and LIF (as control groups).

In the presence of the R2i compound, ICM cells were propagated while maintaining Oct4 expression after 6 days and few trophectodermal cells surrounded the ICM cells. In contrast, in the presence of FBS, regardless of significant ICM outgrowth, the GFP expression decreased over time and disappeared by the 6th day. In this condition, numerous trophectodermal and differentiated cells surrounded the outgrowth of ICM cells.

After about five to seven days, GFP⁺ ICM outgrowths were dissociated enzymatically into single cells and transferred to gelatin-coated plates containing N2B27 medium supplemented with the R2i compound. After six days, the undifferentiated colonies appeared, and were passaged continuously, while expressing Oct4-GFP.

The procedure was extended to mES cell derivation from other mouse strains which are refractory to mES cell derivation under conventional conditions. The R2i compound also supported the efficient derivation of mES cells from DBA/2 and BALB/c with efficiencies ranging between about 50% and 60%.

In order to enhance cloning efficiency, leukemia inhibitory factor (LIF) was added to the medium supplemented with the R2i compound during the mES cell derivation procedure. Leukemia inhibitory factor (LIF) is an important factor of clonogenicity. Therefore, it was seen that presence of the R2i compound and the LIF in the medium supported the highest efficiency for the derivation from different mouse strains including FI of OG2×CD-I, C57BL/6, DBA2, and BALB/c.

Each of the derived mES cell lines were propagated repeatedly in the medium supplemented with the R2i compound as colonies of packed cells with high ratio of nucleus to cytoplasm and obvious nucleoli characteristics of stable mES cell lines. The mES cells which were cultured in the presence of the R2i compound were maintained for over 50 passages, with and without LIF, with no significant changes in growth rate or indications of senescence.

The potential of chimera and germline transmission was also preserved after long-term passages in a medium supplemented with the R2i compound. In addition, Chimerism of Bruce4 mES cells was preserved after 10 passages in a medium supplemented with the R2i compound with initial passage of 18. The obtained data showed that R2i prompt the derivation of mES cells with highest efficiency from different strains and maintain self-renewal and pluripotency along several passaging. In this case LIF could be used optionally for restoring normal population doubling.

### Example 4: In-Vitro and In-Vivo Differentiation

In EXAMPLE 4, the developmental potential of mES cells of EXAMPLE 1 was evaluated by in-vitro and in-vivo differentiation assays. The in-vitro differentiation assay was performed by embryoid body (EB) formation and induced differentiation method. In the EB formation method, mES cells of EXAMPLE 1 were dissociated by adding a trypsin solution to the mES cells. The mES cells were cultured in bacterial dishes including a mES cell medium without leukemia inhibitory factor (LIF). The EBs were subsequently plated onto gelatin-coated plates for about 7 days.

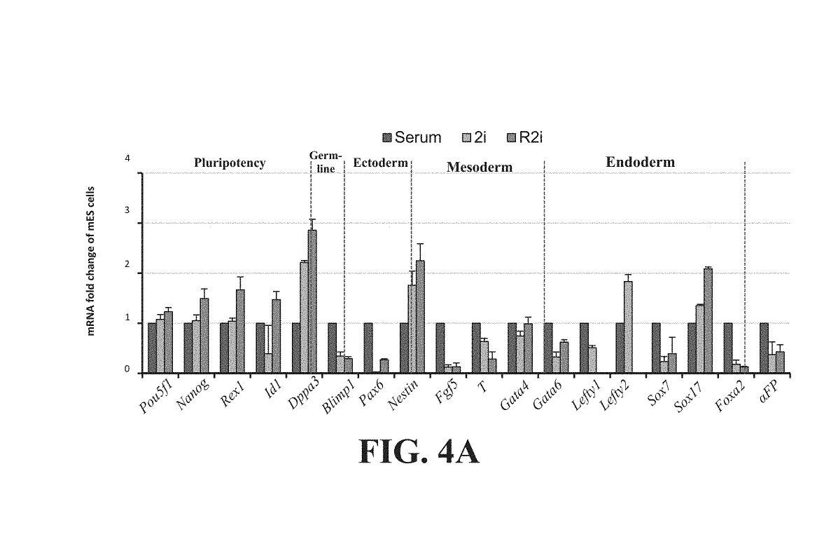

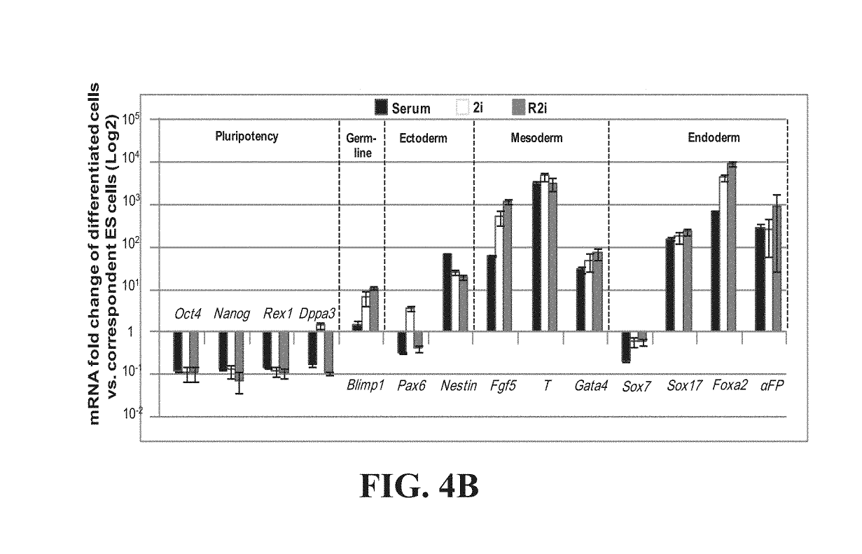

The EBs were then assessed for the expression of pluripotency or lineage-specific genes using a qRT-PCR analysis, and the results were compared with the qRT-PCR results of the mES cells which were cultured at least 7 passages in a medium supplemented with the 2i compound, or with FBS. FIG. 4A illustrates the mRNA fold changes of mouse embryonic stem (mES) cells in different cell culture conditions, according to one or more aspects of the present disclosure. As shown in FIG. 4A, the expressions of pluripotency and lineage-specific genes in mES cells cultured in N2B27 medium supplemented with the R2i compound are higher than the expression of the pluripotency and lineage-specific genes in mES cells cultured in N2B27 medium supplemented with the 2i compound or with FBS.

In the induced differentiation method, the mES cells were differentiated into cardiomyocytes. The mES cells were trypsinized, and cultured at a density of 800 cells/20 gl of hanging drops for 2 days in a mES cell medium without leukemia inhibitory factor (LIF), and supplemented with 0.1 μM ascorbic acid. The embryonic stem cells were then transferred in a bacterial dish for another 3 days and rinsed onto gelatin-coated plates. Beating cells were usually observed between about 1 to 5 days after plating.

Following this process, induced differentiation into neuronal lineages was performed. The 3-day EBs were first transferred to a medium containing 2 μM of retinoic acid for 1 week. They were then plated on gelatin-coated plates and assayed after 3-4 days. For the endoderm lineage differentiation, the EB formation was performed in the N2B27 medium for 3 to 4 days. Following this step, 50 nM of Activin A was added to the EB. After 5 days, the EBs were plated onto gelatin-coated plates and assayed 72 hours later.

The differentiated cells were then assessed for the expression of pluripotency or lineage-specific genes using a qRT-PCR analysis. The results were compared with the qRT-PCR results of the differentiated cells which were cultured at least 7 passages in N2B27 medium with LIF supplemented with the 2i compound or with FBS before performing the induced differentiation. The mES cells cultured in media supplemented with FBS were considered as control. The relative expression levels were normalized to the housekeeping gene GAPDH.

The transcriptional profile of mouse embryonic stem (mES) cells do not exhibit fixed state and could be interconvertible after transfer of mES cells which were cultured in a medium supplemented with the 2i compound to a medium supplemented with the serum and vice versa. FIG. 4B illustrates the mRNA fold changes of differentiated cells with respect to corresponding mouse embryonic stem (mES) cells, according to one or more aspects of the present disclosure. As shown in FIG. 4B, expression of pluripotency and lineage-specific genes are higher in the mES cells which were cultured in N2B27 medium supplemented with the R2i compound than expressions of pluripotency and lineage-specific genes in the mES cells which were cultured in N2B27 medium supplemented with the 2i compound.

Furthermore, RT-PCR analysis showed the ability of formed EBs to generate the derivatives of three embryonic germ layers. Lineage specific differentiation protocols exhibited the capability of R2i cells to form Map2⁺ and Tujl⁺ neuronal lineage, GATA4⁺ mesoderm, Mhc⁺ cardiomyocyte, and Foxa2⁺ and αFP⁺ endoderm. The data revealed that despite the lowest level of most of the lineage-specific genes of mES cells which were cultured in medium supplemented with the R2i compound, differentiation events presage properly after removing the R2i compound from the medium. Moreover, transcriptional profiles of archetype stem cell markers and lineage-specific genes confirmed that the mES cells which were cultured in the medium supplemented with the R2i compound exhibited bona fide ground state of pluripotency.

Referring again to FIGS. 4A and 4B, high pluripotency-affiliated gene expression and low to absent of the most line-specific gene profile showed that R2i support the stable “ground state” in mES cells. The R2i significantly reduced the expression of various developmentally regulated genes, including Blimp1, Pax6, Fgf5, Brachyury, Lefty1, Lefty2, Sox7, Foxa2, and αFP. Although the unstable expression of pluripotency and lineage-specific genes can be understood to result from the intrinsic ability of mES cells to beget differentiation into specified lineages, these conditions are usually resolved with serum that would not be considered optimum culture conditions.

Moreover, the mES cells which were cultured in a medium supplemented with the R2i compound are homogenous in terms of the expression of pluripotency markers such as Nanog and Stella. These mES cells which were cultured in a medium supplemented with the R2i compound have less differentiation leakage (for example, expression of lineage differentiation marker genes such as Lefty 1, Lefty 2, and Brachyury) in comparison with serum or a medium supplemented with the R2i compound.

In order to perform a teratoma formation analysis, 3×10⁶ to 5×10⁶ mES cells were re-suspended in Matrigel and injected subcutaneously into syngeneic mice, while the growth of the teratoma was monitored. Paraffin sections of tumor masses were stained with hematoxylin and eosin (H&E) for all histological determinations. The differentiation capacity of R2i cells was further assessed using chimera formation in colored C57BL/6 and DBA2 derived mES cells.

All of the selected mES cell lines contributed to the chimera, including the germline. In order to generate the chimeric mice, dissociated mES cells were injected into the E 3.5 blastocysts. The chimeric mice were generated by standard protocol. Chimerism was determined by coat color in mice. The mES cells which were cultured in a medium supplemented with the R2i compound demonstrated the capability of chimera formation. In addition, germ-line transmission was tested as the chimeras were mated to the males or females from mouse strains containing injected mES cells. It was shown that mES cells which were cultured in a medium supplemented with the R2i compound can differentiate properly by induced differentiation, chimera formation, and germline formation.

### Example 5: Flow Cytometry Analysis

In EXAMPLE 5, the mouse embryonic stem (mES) cells that were cultured in media supplemented with R2i compound or with the 2i compound were subjected to a flow cytometry analysis. The trypsinized embryonic stem cells were fixed with ice-cold methanol and blocked by 2% normal goat serum for 60 minutes, washed, and incubated with primary antibody (Oct4, Nanog, and Stella) for 1 hour at 37° C. The mES cells were then washed again and treated with secondary antibody for 30 min at 37° C. After a final washing, a flow cytometry analysis was performed using the Fluorescence-activated cell sorting (FACS) calibur flow cytometer. The acquired data was analyzed using the BD CellQuest Pro software.

As a negative control, the cells were stained with the appropriate isotype-matched control. In order to evaluate homogeny of mES cells in a medium supplemented with the R2i compound, two mES cell lines of Royan B20 and Royan D4 were cultured in media including serum +LIF, 2i±LIF, and R2i±LIF for 7 passages. In addition, protein expression profiles of these mES cell lines were examined with respect to Nanog and Stella expression by flow cytometry.

### Example 6: Karyotype Analysis

Displaying a stable karyotype during repetitive passages of mouse embryonic stem (mES) cells is one of the more challenging issues in developing a culture medium. Although the use of small molecule inhibitors in a defined medium has advanced the derivation of pluripotent stem cells through rodentia, there is a concern that these chemical perturbations may threaten genomic integrity of cells.

The application of CHIR99021 small molecule in a medium supplemented with the 2i compound gives rise to an apprehension of chromosomal abnormalities heightening in mES cells. This is due to GSK3 inhibition by related small molecules or RNA interference that endangers chromosomal alignment at mitosis. In order to evaluate the effects of the R2i compound on mES cell culture and compare the effects of the R2i compound with the 2i compound, the karyotype of 7 selected virtually normal mES cell lines after long-term passaging in media supplemented with the R2i compound or the 2i compound was assessed.

In EXAMPLE 6, a chromosomal integrity test and a karyotype analysis were performed. The mES cells of EXAMPLE 1 were first treated with thymidine at a concentration of about 0.66 mM for about 12 hours at a temperature of about 37° C. The mES cells were washed with a fresh medium. After 4 hours, the mES cells were treated with Colcemid at a concentration of about 0.15 mg/ml for about 30 minutes.

In the next step, the mES cells were trypsinized and swelled using a potassium chloride (KCl) solution at a concentration of about 75 mM for about 15 minutes. The mES cells were then fixed in ice-cold methanol and acetic acid mixture with a ratio of about 3:1 (volume of methanol: volume of acetic acid), and dropped onto chilled slides. The chromosomes were then visualized using a standard G-band staining, and 50 chromosomes at a metaphase stage were screened by a microscope and counted on micrographs.

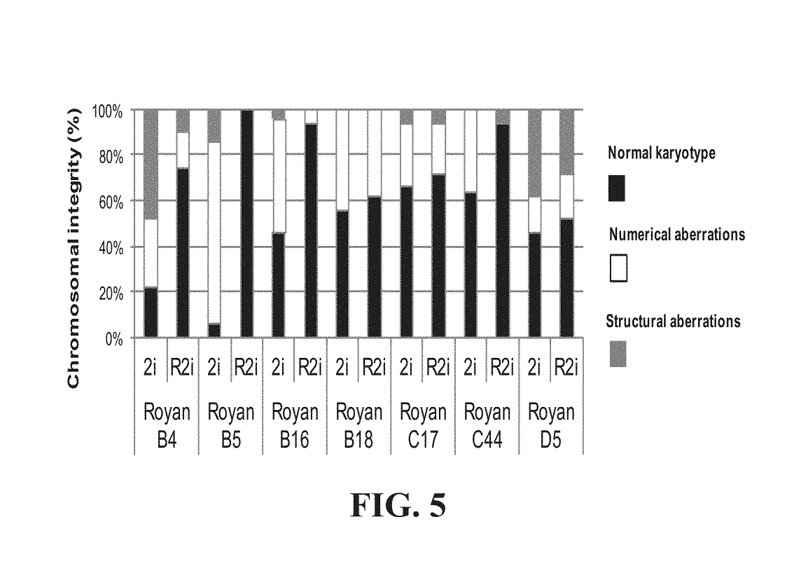

FIG. 5 illustrates a chromosomal integrity profile of mES cells after a long-term cultivation in a medium supplemented with the R2i compound or with the 2i compound, according to one or more aspects of the present disclosure. Referring to FIG. 5, it can be seen that the medium supplemented with the R2i compound supports the maintenance of normal karyotype more efficiently, and two-fold more than the medium supplemented with 2i compound, after at least 20 passages of different mES cell lines in a medium supplemented with the 2i or the R2i compound.

The data of FIG. 5 further shows that while mES cells in the medium supplemented with the 2i compound may undergone many alternations, the medium supplemented with the R2i compound could retain a normal diploid karyotype until high passage number. Therefore, the R2i compound does not cause chromosomal aberrations and can be understood to serve as a reliable substitution for the 2i compound in cultivation of mES cells.

### Example 7: Establishing Pluripotency in Mouse Embryonic Stem Cells Using TGF-β Inhibitors

In EXAMPLE 7, pluripotency was maintained in mES cells using different inhibitors of the TGF-β signaling pathway, such as SB431542 small molecule, A83-01 small molecule, ALK5i small molecule, siRNAs against Smad2 molecule, and siRNAs against Smad3 molecule.

In order to inhibit TGF-β signaling pathway using siRNAs, three different siRNAs were used for knocking down each of Smad2 and Smad3. The mES cells were transfected with the siRNAs, and the medium was changed 24 hours and 48 hours after the transfection of the mES cells. The mES cells were then collected, and subjected to an mRNA isolation with a standard protocol. The isolated mRNA was subjected to qRT-PCR analysis. TABLE 7 below presents the sequences of the siRNAs against Smad2 and Smad3.

TABLE 7
Sequences of siRNAs against Smad2 and Smad3
siRNAs against Smad 2

| Name | Sense sequence | Anti-sense sequence |
|---|---|---|
| siRNA A | SEQ ID No. 1 | SEQ ID No. 2 |
| siRNA B | SEQ ID No. 3 | SEQ ID No. 4 |
| siRNA C | SEQ ID No. 5 | SEQ ID No. 6 |
| siRNA D | SEQ ID No. 7 | SEQ ID No. 8 |
| siRNA E | SEQ ID No. 9 | SEQ ID No. 10 |
| siRNA F | SEQ ID No. 11 | SEQ ID No. 12 |

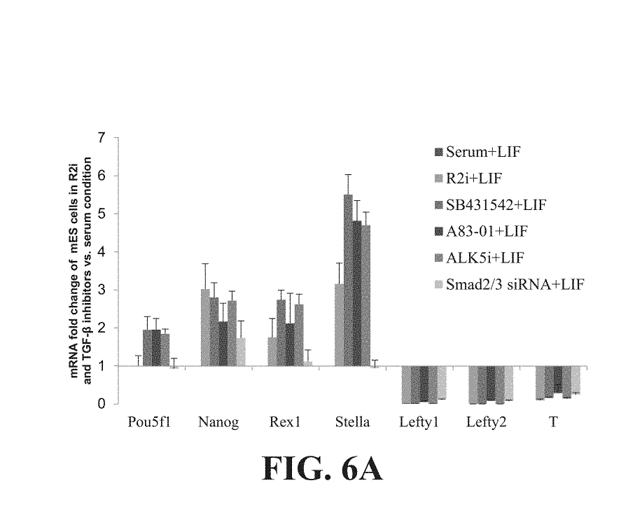

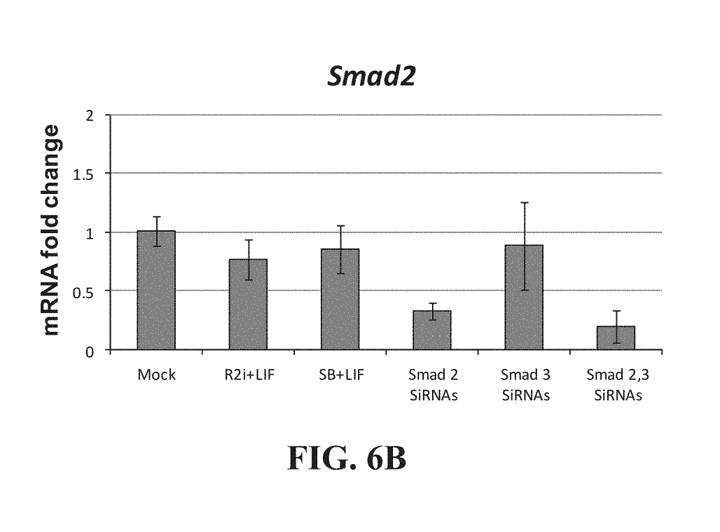

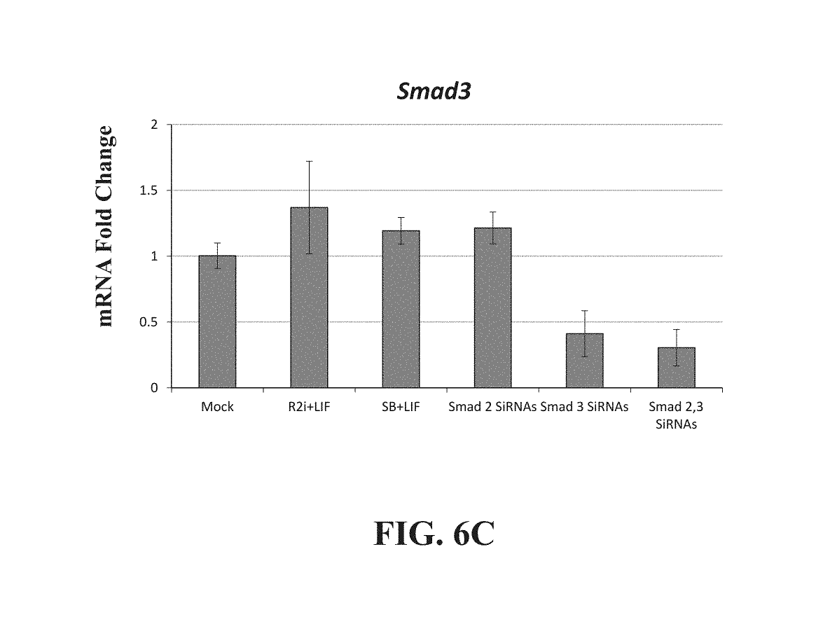

FIG. 6A illustrates the mRNA fold change of pluripotency and differentiation-specific genes of mES cells which were cultured in different culture conditions versus the serum condition. The mRNAs which were used for qRT-PCR analysis were Oct4, Nanog, Rex-1, Dppa3, Lefty 1, Lefty 2, and T. The relative expression levels of the genes were normalized to the housekeeping gene GAPDH.

The qRT-PCR analysis was done for Royan B20 mES cell line after seven passages in media supplemented with TGF-β inhibitors, such as SB431542, A83-01, and ALK5i. In some groups, Smad2 or Smad3 molecules of the mES cells were knock-down using the siRNAs. The serum was considered as control, and the media were supplemented with LIF.

Referring to FIG. 6A, it can be seen that the mRNA fold change in the mES cells is minimal in the presence of the R2i compound. The qRT-PCR analysis confirmed that different TGF-β small molecule inhibitors or Smad2 and Smad3 siRNAs (all with LIF), maintain the pluripotency in mouse mES cell culture after several passaging in N2B27 defined medium. However, it can be understood that a medium supplemented with the TGF-β inhibitors and LIF would not be considered an optimum culture medium for mES cells. For example, this condition does not lead to mES cell line derivation and is also unable to form undifferentiated colonies from isolated single mES cells. As shown in FIG. 6A, the data confirms that inhibition of TGF-β signaling supports the pluripotency in mES cells.

FIG. 6B illustrates mRNA fold changes of pluripotency markers of the mES cells in which the Smad2 molecule was knocked down by use of siRNA molecules, according to one or more aspects of the present disclosure. FIG. 6C illustrates mRNA fold changes of the mES cells in which the Smad3 molecule was knocked down by use of siRNA molecules, according to one or more aspects of the present disclosure. As shown in FIGS. 6B and 6C, knock-down of the Smad2 and Smad3 molecules is associated with the same effects as using the R2i compound in the expression of pluripotency markers.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for derivation and maintenance of pluripotency in mouse embryonic stem (mES) cells, the method comprising:
isolating mES cells from mouse embryos in a culture medium including an R2i compound, wherein the R2i compound includes a combination of a transforming growth factor beta (TGF-β) signaling pathway inhibitor and an extracellular signal-regulated kinases (ERK) signaling pathway inhibitor; and
culturing the mES cells in a medium including the R2i compound or the TGF-β signaling pathway inhibitor.

2. The method according to claim 1, wherein isolating mES cells from mouse embryos further comprises:
removing zona pellucida from the mouse blastocysts;
culturing zona-free blastocysts in a culture medium including the R2i compound; and
dissociating cells of the cultured zona-free blastocysts to obtain mES cells.

3. The method according to claim 1, wherein the TGF-β signaling pathway inhibitor includes a small molecule, or a siRNA molecule.

4. The method according to claim 3, wherein the small molecule is selected from the group consisting of SB431542, A83-01, ALK5i, or combinations thereof.

5. The method according to claim 4, wherein the SB431542 small molecule is present in the culture medium at a concentration ranging between approximately 2 μM and 10 μM.

6. The method according to claim 4, wherein the A8301 small molecule is present in the culture medium at a concentration of approximately 0.5 μM.

7. The method according to claim 4, wherein the ALK5i small molecule is present in the culture medium at a concentration of approximately 1 μM.

8. The method according to claim 3, wherein the siRNA molecule is an inhibitory molecule against Smad2 molecule, Smad3 molecule, and combinations thereof.

9. The method of claim 1, wherein the ERK signaling pathway inhibitor includes a small molecule.

10. The method of claim 9, wherein the ERK signaling pathway inhibitor is PD0325901.

11. The method according to claim 10, wherein the PD0325901 is present in the culture medium at a concentration of approximately 1 μM.

12. The method according to claim 1, wherein the mouse embryos includes blastocysts, inner cell mass (ICM) cells, or single blastomeres cells.

13. The method according to claim 12, wherein the single blastomere cells are isolated from 2-cell embryos, 4-cell embryos, or 8-cell embryos.

14. The method according to claim 1, wherein the mouse embryonic stem cells are obtained from a mouse strain which is selected from a group consisting of NMRI strain, C57BL/6 strain, BALB/c strain, DBA/2 strain, and a hybrid of C57BL/6 strain and CD-1 strain.

15. The method according to claim 1, wherein the culture medium includes a serum-free medium.

16. The method according to claim 15, wherein the serum-free medium includes one of N2B27 defined medium, or knock-out serum replacement (KoSR) supplemented medium.

17. The method according to claim 1, wherein the culture medium further comprises leukemia inhibitory factor (LIF).

18. The method according to claim 1, wherein the steps of isolating and culturing the mES cells occur in an adherent culture or a suspension culture.

19. The method according to claim 18, wherein the adherent culture includes a feeder-free culture.

20. The method according to claim 1, wherein the mES cells are passaged for a period of time ranging between about 2 days and 3 days in the culturing step.
